(19) United States
(12) Patent Application Publication
(10) Pub. No.: US 2009/0017092 A1
(43) Pub. Date: Jan. 15, 2009

(54) Novel Class of Cell-Interactive Material and Process of Preparation of Artificial Tissues of Human and Animal Origin

(75) Inventors: Aroop Kumar Dutta, Lucknow (IN); Ranjna Dutta, Lucknow (IN)

Correspondence Address: THE WEBB LAW FIRM, P.C., 700 KOPPERS BUILDING, 436 SEVENTH AVENUE, PITTSBURGH, PA, 15219, US

(73) Assignee: Aroop Kumar Dutta, Lucknow (IN)

(21) Appl. No.: 12/171,910

(22) Filed: Jul. 11, 2008

(30) Foreign Application Priority Data
Jul. 12, 2007 (IN) 1468/DEL/2007

(51) Int. Cl. C12N 5/08; C12N 5/06; A61K 35/12; A61F 2/00

(52) U.S. Cl. 424/423; 435/397; 424/93.7

(57) ABSTRACT

An artificial extra cellular matrix product and artificial human and animal tissue product comprising tissue specific cells.

### FIELD OF INVENTION

This invention relates to a novel class of artificial extra cellular matrix and a process for the preparation of the same.

### BACKGROUND OF THE INVENTION

The art of creation of an artificial tissue product can described conventionally as two step process. Step one, is the creation of a three dimensional scaffold with interconnected pores of some biocompatible material and step 2, is the subsequent addition of desire type of cells for culture. (Trends Biotechnol. 17 pp 409, 1999; Curr. Opin. Biotechnol, 10 pp 123-129, 1999).

Creation of a three dimensional scaffolds as artificial extra cellular matrix using collagen, gelatin, glycosaminoglycan, other macromolecules of extra cellular matrix origin as well as synthetic polymers like poly lactic acid etc. has allowed simple tissue formation by incorporating desired kind of cells. Artificial skin, cartilage, urinary bladder etc. are examples of such tissues (Autologous cell transplantation for urologic reconstruction. J. Urol. 159 pp 2-3, 1998; Design of an artificial skin. I. Basic design principles. J. Biomed. Mater. Res., 14 pp 65-81, 1980; Tissue engineering of a bioartificial tubule ASAIO J., 44 179-183, 1998 and Z Rheumatol 58 pp 130-135, 1999)

For example, artificial skin has been formed by creating a porous scaffold of collagen and keratan/dermatan sulfate by freeze drying and crosslinking the scaffold molecules together. Cells of skin origin like epithelial cells, keratinocytes etc. can then be incorporated by means of superficial inoculation and culturing for weeks the cells and scaffold together till a three dimensional thick layer of cells is formed on the scaffold.

The prior art processes suffered from several disadvantages. Some of the important drawbacks as follow:
1. Current biocompatible material of synthetic origin as porous three dimensional scaffold are passive towards cell and do not have cell interactivity like extra cellular matrix present in the natural tissue.
2. Artificial extra cellular matrix reconstituted from individual components of extra cellular matrix have good cell interactivity but very poor engineering properties. Therefore, any fabricated device reconstituted from individual component of extra cellular matrix is extremely delicate and difficult to handle;
3. Subsequent addition of cells on the porous scaffold need to be properly distributed in the three dimensional scaffold and incubated for at least a few week in an incubator to attain a minimum number of cells by growth before it can be considered as a tissue equivalent of artificial origin. Both factors namely, cell distribution and extent of growth is critical to the formation of a tissue equivalent;
4. Subsequent formation of artificial tissue with cells and porous scaffold has an upper limit on thickness due to the diffusive limitation of oxygen and nutrition availability to cells growing deeper inside the pores of scaffold. Cells of skin and cartilages and other non/less vascularized tissues particularly can grow under such limitations due to their lower oxygen and nutrition requirements and can form artificial tissue of 1-5 millimeter thickness;
5. Most natural tissues have vasculature [e.g. arteries and veins] to support realistic tissue formation with thickness beyond a few centimeter to support multilayer (three dimensional) of cells with appropriate amounts of nutrition and oxygen through the circulation of fluids [e.g. blood] in vasculature. Further, such vasculature also help in removing toxic end products like ammonia, lactic acid, carbon dioxide etc. formed by consumption of nutrition and oxygen by the cells. Briefly, without vasculature it is impossible to form an artificial tissue with thickness beyond a few millimeters;
6. Current artificial tissues incorporate a minimum variety of cell types, generally a single type or two to three types of cells in case of full thickness skin, whereas a natural tissue can be visualized as an organized cluster of many types of cells along with extra cellular matrix which in turn is an highly ordered assembly of macromolecules specific to the tissue.
7. Porous scaffolds fabricated using synthetic polymers involve many toxic chemicals that need to be removed before it is mixed with cells to create artificial tissue.
8. Artificial tissue created by growing cells in porous three dimensional scaffold need to be grafted by invasive surgery depending upon shape and size of the scaffold.

### SUMMARY OF THE INVENTION

An object of the present invention to provide a three dimensional porous scaffold from components of extra cellular matrix with or without cells for the preparation of artificial tissue product, which does not involve use of toxic chemicals.

Further object of the present invention to provide a three dimensional porous scaffold from components molecules of extra cellular matrix which have good cell interactivity like extra cellular matrix present in the natural tissue and yet evince good range of engineering properties allowing convenient fabrication of devices with desirable strength and shapes.

Yet another object of the present invention to provide a three dimensional porous scaffold from component macromolecules of extra cellular matrix which envisages improved cell distribution and extent of growth.

Another object of the present invention to provide a process for the preparation of three dimensional porous scaffold from component molecules of extra cellular matrix which overcomes various drawbacks of the prior art products.

### BRIEF DESCRIPTION OF THE DRAWINGS

FIG. 1 shows the microcarier parts described in Example 2 of the present invention, as observed under an optical microscope;

FIG. 2 is directed to an ear like prosthesis ready for culture with culture cells produced by methods of the present invention;

FIG. 3 is a histological section directed towards in vitro cultures of externally inoculated CHO-K1 cells such as those described in Example 5 of the present invention;

FIG. 4 is a histological transverse sectioning of a blood vessel in the injection emulsion gel without cells;

FIG. 5 is a histological transverse sectioning of a blood vessel in the injection emulsion with cells; and

FIG. 6 is a histological longitudinal sectioning of a blood vessel in the injection emulsion with cells.

### BRIEF DESCRIPTION OF THE INVENTION

According to this invention there is provided a novel artificial extra cellular matrix product and artificial human and animal tissue product with tissue specific cells.

In accordance with this invention there is provide a process for the preparation for artificial extra cellular matrix and artificial human and animal tissue product comprising:

Making an oil in water emulsion from water solution of extracellular matrix molecules, other constituents as required with fluorocarbon and a biocompatible emulsifier;
subjecting the said emulsion to the step of crosslinking to form an emulsion gel; optionally, adding desired cells prior to the addition of cross-linker and mixing cells to distribute the cells in the emulsion thoroughly;
Shaping the emulsion gel to form a device;

### DETAILED DESCRIPTION OF THE INVENTION

This invention provides a process to create a three dimensional porous scaffold from component macromolecules of extra cellular matrix with or without cells for the preparation of artificial tissue product. It also provides method of formation of devices by one or more types of operations but not limited by the way of example like molding, extrusion, casting, printing and other means.

The disclosed method does not have limitations as mentioned earlier typical to conventional processes of creating artificial tissue and porous three dimensional scaffold products.

The method involves a micro-emulsion, macro-emulsion or mixed emulsion of aqueous solution of macromolecules of extra cellular matrix origin with an organic phase as may be desired to create porous scaffold. Emulsion can be further processed by gelling it by crosslinking with a crosslinking enzyme e.g. transglutaminases. Before the emulsion sets-in permanently as gel, it can be processed through one or more manner of handling but not limited by the way of example such as extrusion, casting, molding, coating, printing as required for the fabrication of specific devices.

Cells of more than one variety can be added in this emulsion prior to crosslinking or soon after, before further processing the emulsion. Cells can also be added after the final processing step of creating a three dimensional porous scaffold of desired shape and size.

A brief list of individual components but not limited to as example, for the formation of aqueous solution is as follows; macromolecules of extra cellular matrix origin in pure or crude forms like gelatin, collagens, fibrin, fibrinogen, fibronectin, solubilized elastin, laminin, glycosaminoglycan, proteoglycan, glycans, hyaluronic acid. Biodegradable additives of synthetic origin like (but not limited to) polyvinyl alcohol, polyvinyl pyrrolidone, poly ethylene oxide, cellulose or starch derivatives can also be added to fine tune the rheological and physical properties of the emulsion. Certain electrolytes based buffers can also be used to prepare the solution.

Emulsion of the above solution is created by using non toxic fluorocarbons, silicone oils and higher liquid hydrocarbon and surfactants. If interconnected macro-pores are desired for the preparation of porous three dimensional scaffolds only (not a tissue equivalent with cells), a macro-emulsion of silicone oil/hydrocarbon is more suitable and economical.

Crosslinking step to render this emulsion permanently as gel can be achieved by enzymatic means. Transgutaminases of microbial or animal origin and/or factor XIII from human or animal blood or from guinea pig liver and/or lysyl oxidase can be used in appropriate quantities.

The method of this invention is an improvement over an earlier patent (Indian Patent No. 191148) where all toxic ingredients (organic solvents and chemical emulsifiers), harsh physical and chemical steps (like chemical crosslinking, washing with organic solvents, autoclaving etc.) have been removed and replaced by biocompatible non toxic and physiologically compatible steps.

Cell-biomaterial interaction of artificial extra cellular matrix is also improved by incorporating other extra cellular matrix components without inactivating/denaturing them and allowing them to assemble in a similar manner they are assembled in a natural extra cellular matrix.

These modifications avoiding toxic or harsh processing conditions allow direct addition of cells least compromising their viability, functionality and unwanted differentiation by adequate supply of nutrition, oxygen and other factors.

A tissue of an organ consists of sub structures of varying cell types and extra cellular matrix. Extra cellular matrix component macromolecules bind with the cells and help them to maintain their normal biological functions. Cells in turn produce different components of extra cellular matrix depending upon the cues from environment and genetic make up. This dynamically active complex of cells and extra cellular matrix forms the basis of physical and biological functions of all tissues and organs (normal or pathological). (Weaver, V. M. et al. J. Cell Biol., 137 pp 231-245, 1997 and Cukierman, E., et al. Science, 294 pp 1708-1712, 2001).

In order to create a realistic tissue like function artificially, it is essential to support cells in a three dimensional manner, aided by a porous three dimensional scaffold made of a biodegradable polymer (Trends Biotechnol., 16 pp 224-230, 1998 and Nature 424 pp 870-872, 2003). Cells further need to be cultured in a liquid medium consists of nutrition and dissolved oxygen. Cells that are deeper inside the scaffold may not get same amount of nutrients as cells present close to external surface of the scaffold, With increasing scaffold thickness and cell number, it is exceedingly difficult to provide nutrients to cells inside the scaffold. It is impossible to create a tissue beyond thickness of a few millimeters with as many cells present in a natural tissue on a porous scaffold due to these limitations.

In a naturally occurring animal tissues a three dimensional stacking of cells is maintained by vascularization (provision of arteries, veins and other luminal structures) to supply nutrients and oxygen as well removal to toxic waste materials produced by the cells.

Reconstituted extra cellular matrix based three dimensional scaffolds like collagen gels, matrigel, hubiogel on the other hand are capable of providing appropriate level of interactivity by binding with the cells and maintain the cell function for longer period. However, they do not have appropriate physical properties to create practical devices due to physically and chemically delicate nature of constituent molecules and gels created from these constituents.

Moreover, porous scaffold currently made of polylactic acid, polyglycolic acid, poly caprolactone, polyhydroxy butyric acid and other synthetic polymers do not act as cementing material between cells by binding with them, however provide a biocompatible passive support that does not allow cells to maintain their normal functions over extended time period (Ann. N.Y. Acad. Sci., 961 pp 10-26, 2002).

This invention provides a method to overcome all of the critical limitations in a three step process to create an artificial tissue product.

First step, the porous scaffold with extra cellular matrix mimicry is made from an emulsion of extra cellular matrix constituents as required with fluorocarbon (droplet size of fluorocarbon is 0.05 to 0.25 micron, U.S. Pat. No. 4,252,827) and a biocompatible emulsifier. Presence of fluorocarbon allow sufficient amount of oxygen due to high oxygen solubility in fluorocarbons. An emulsion with the help of a biocompatible emulsifier will ensure proper distribution of fluorocarbon leading to oxygen delivery to the interiors of the scaffold.

In the second step, a biological cross-linker like transglutaminase or lysyl oxidase is used to crosslink the emulsion as permanent emulsion-gel in a physiologically compatible condition to crosslink the emulsion as emulsion-gel.

Optionally, cells of a desired tissue are added just prior to the addition of cross-linker or soon after and mixed well to distribute the cells in the emulsion thoroughly.

Finally, the third step is shaping the emulsion gel in the format of a device and incubating the emulsion in a physiologically compatible condition to allow cells to proliferate or function in a desirable manner.

The process of preparation of said emulsion of the first step is explained hereafter.

Oil (organic phase) in water type of emulsion with amounts of oil higher than water is an unusual emulsion gel (J. Phys Chem., 90 pp 5892-5895, 1986; J. Chem Soc. Chem. Commun., pp 1807-1809, 1989) that allows higher volume of oil phase held by network of water and soluble protein in a continuous or bi-continuous emulsion of possibly Winsor III type region, Stability of this emulsion gel is due to highly tensed water-protein network that is held in place by highly ordered inter molecular hydrogen bonds within protein network (of aligned collagen folds) and further stabilized by emulsifiers.

Due to a tensile water-protein phase the viscosity of emulsion of this invention increases with increasing amount of fluorocarbon/hydrocarbon. If it is not desirable to use fluorocarbon in high amounts, silicone oil or saturated hydrocarbon like paraffin oil can also be used. However, dissolved oxygen carrying capacity of fluorocarbon is not matched by these substitutes. These examples of oil phase when mixed with water solution of biologically functional molecules like growth and differentiation factors, enzymes and proteins do not denature them.

Emulsion can be prepared by sterile constituents under aseptic conditions if it is desirable to perform cell culture or grafting on animal or a therapeutic implantaion.

The total concentration of extracellular matrix components in solution and optional components can vary from 1% to 20% by weight to the volume of water. Proteins of extra cellular matrix origin are particularly suitable for the emulsion however any protein that can be dispersed in water phase as long as it is suitable substrate for transglutaminase or lysyl oxidase crosslinking can be used. Some examples of the protein but not limited as examples are gelatin, collagens, laminins, fibrinogen, fibronectin, elastin and keratin.

Optional additives can be added to the protein solution to enhance cell-scaffold interactivity and rheological properties like (but not limited as examples), proteoglycans, hyaluronic acid, glycosaminoglycans, growth factors (insulin like growth factor, platelet derived growth factor, transforming growth factors, fibroblast growth factors, epithelial and endothelial growth factors, angiogenic factors, neuronal growth factor, stem cell growth factor, bone morphogenic proteins), antioxidant enzymes like superoxide dismutase, nutrition transporters like insulin and transferring, small molecules like polyamines (glutathione, spermidine, putresceine, cadaverin).

Biodegradable additives of synthetic origin like (but not limited as examples), polyvinyl alcohol, polyvinyl pyrrolidone, and polyethylene oxide can also be added to fine tune the rheological properties of emulsion and physical properties like strength and flexibility of three dimensional porous scaffolds.

To create bone like extra cellular matrix, solution can be mixed with following materials but not limited as example; calcium salts like calcium sulfate, tricalcium phosphate, hydroxyapatite, bone putty, and non mineral components of bone like demineralized bone matrix, bone morphogenic proteins.

Concentration of organic phase may vary from one tenth to ten times the volume of water-protein phase. Oil phase may consist of one or more types of fluorocarbons, silicone oil (poly dimethyl siloxane) and saturated hydrocarbons like liquid paraffin.

Organic phase consisting of one tenth to ten times by volume of the water solution emulsified within the water solution using a physiological emulsifier and other additives. Organic phase is one or more fluorocarbons but not limited as examples, like; octafluoropropane, perfluorooctane, perfluorohexane perfluorodecaline, perfluorotripropylamine, perfluoromethyidecalin, perfluorobutyltetrahydrofuran, perfluoroperhydrophenanthrene, perfluorobutane, perfluoropolyethers, hydrofluoropolyethers, halide, sulfur, phosphate, amine, ether containing derivatives or silicone oil like polydimethylsiloxane or liquid saturated hydrocarbon like paraffin oil or a mixture of these.

Suitable emulsifier to create emulsion of fluorocarbon with water-protein solution can be but not limited as example like, bile salts, phospholipids, lecithins, pulmonary surfactant proteins, glycosaminoglycans, fatty acids, and triglycerides at a concentration of 10 to 100000 milligram per liter of emulsion. These surfactants are normally present in the animal body and do not disturb cells in harmful manner under physiological concentrations or conditions.

Synthetic emulsifiers like but not limited as example, semifluorinated alkanes, fluorinated surfactants, phosphorous fluorinated surfactants, polyoxyethylene polyoxypropylene copolymer (Proxanol 268), pluronic F68, polypropylene oxide, polyethylene oxide can be used for the preparation of emulsions, however their application for medical purpose need to be thoroughly established as in many cases there is no prior knowledge for their safety and efficacy.

Cell concentration that needs to be added can be adjusted according to situation like how fast an artificial tissue need to functional and the availability of the desired kind of cells. Typically 0.01 million to 100 million cells per milliliter of emulsion can be added. However, even higher number of cells may be desired in specific situation and can be incorporated in the emulsion without much difficulty. Cells can be of single type or multiple types found in a specific tissue/organ. After addition of the cells a gentle mixing is needed to ensure homogeneous distribution of cells in the emulsion.

It is essential to provide physiological conditions and nutrition to the cells in case they are to be added in the emulsion to keep them in healthy condition. An appropriate composition of salt solution for pH buffering, minerals, amino acids and vitamins are added in the emulsion prior or along with the cells.

Cells of a tissue in the form of suspension in sterile media can be mixed in this emulsion before or soon after the addition of sterile transglutaminase or lysyl oxidase solution to crosslink this emulsion.

Transglutaminases for use in the present invention can be prepared according to the known means including microbial fermentation (J Food Chem. 27 pp 109-125 2003 or U.S. Pat. No. 5,156,956), extraction from plasma and tissues (Cooke and Holbrook Biochemical J 141 pp 7-84, 1974 and Curtis and Lorand Methods Enzymol. 45 pp 177-191, 1976) or by recombinant yeast fermentation (Bishop et al. Biochemistry 29 pp 1861-1869, 1990).

Lysyl Oxidase for use in the for use in the present invention can be prepared according to the known means including animal tissue extract (J Biol. Chem 251 pp 5779-5792, 1976) or a recombinant source (FEBS Letters 399 pp 215-219, 1996).

Transglutaminase at a concentration of 0.01% to 1% by weight to the volume of the emulsion is used for crosslinking of the emulsion to a permanent firm gel under appropriate condition of incubation. Similarly, lysyl oxidase can be used in presence of traces of copper ion in the concentration range 0.01% to 1% weight by volume depending upon the purity, and time of crosslinking required for the purpose.

Incubating the emulsion with or without cells under physiological conditions further comprise of conditions essential for crosslinking the emulsion, like presence of 1-20 mM (preferably 2-5 mM) calcium chloride, thrombin 0.1-10 units/mL (preferably 1 unit/mL), at a temperature of 2° C. to 40° C. (preferably 250 to 37° C.) and a pH or 5.5 to 8 (preferably 6.5 to 7.5). Physiological buffer consists of a salt solution with buffering agents, minerals and nutritional agents like glucose, amino acids and vitamins.

Immediately after the addition of transglutaminases or lysyl oxidase and cells (if required), the emulsion is shaped in to required form by extrusion, molding, casting, coating or printing in the form of final device. For example, small spherical shape of about 500 micron is given for large scale cell culture purpose in a suspension bioreactor. Shape of an organ like, ear and nose cartilage or any other shape like disc, cylinder or cubes can also be given to augment the cavity generated by any injury in cartilage tissues. A sheet like film of the emulsion can be spread over the damaged skin or molded separately and then applied on the damaged skin area. A pattern can be printed in 3 dimensions by jet printers for rapid prototyping with intricate patterns with multiple emulsions containing specific cell types.

Petri plates, multiwell plates and any other glass and plastic surface can be coated with this emulsion containing transglutaminase in a manner of predefined three dimensional patterns for cell culture in laboratory to create cultured equivalents of tissues as research, diagnostic and investigative tools.

Further, this emulsion can be brought in contact with sheet of porous polymer film consisting of either a non woven fabric, cellulose based papers, screen and meshes made of silicone, biodegradable polymer like poly lactic acid, polygycolic acid, polyhydroxy butyric acid, poly caprolactone and other natural polymers like alginic acid and chitosan.

Injecting the emulsion or grafting the emulsion scaffold with or without cells in an animal at the site of injury to improve its function. The emulsion-gel scaffold is capable of inducing vascularization in the emulsion scaffold and sustains the cells alive for long periods as shown in the example.

All the process steps can be performed in a sterile manner including the incorporation of sterile ingredients particularly if cells are added in the emulsion to avoid sepsis due to contamination by bacteria and other microorganisms. Otherwise final three dimensional scaffold devices without cell can be sterilized by radiation.

This method is an improvement of an earlier patent (Indian Patent No. 191148) where all toxic ingredients (organic solvents and chemical emulsifiers), harsh physical and chemical steps (like chemical crosslinking, washing with organic solvents, autoclaving etc.) have been removed and replaced by non toxic and physiological compatible steps. Advantages of this method and how exactly limitation of the earlier and existing methods are overcome will be explained hereafter.

This method allows incorporation of varied components of extra cellular matrices to mimic the cell-matrix interaction of a specific tissue without structurally damaging or denaturing them. Other additives and crosslinking of viscous emulsion ensure sufficient mechanical and engineering properties to create products of desirable capabilities.

Since cells can be mixed during the process of emulsion formation without harm allowing it to be homogeneously distributed through out the scaffold (emulsion), it is not necessary to inoculate cell externally leading to insufficient distribution and occupancy of inner most spaces of a formed scaffold. Emulsion with cells and transglutaminase or lysyl oxidase can be directly used by injecting or applying at the site of injury thereby avoiding in vitro culture step as long as three to four weeks completely.

Use of fluorocarbons in the emulsion making has dual purpose. Firstly, it creates enough internal void space for cells to spread and allow nutrient to be transported. Secondly, fluorocarbon as micro-emulsion also deliver sufficient amount of dissolved oxygen to the cells that are present deep inside the emulsion scaffold.

This ability to deliver sufficient nutrition and oxygen create realistic tissue like cell assembly and ultrastructures of relatively large sizes.

Cell-biomaterial interaction is also improved by incorporating other extra cellular matrix components without inactivating/denaturing them and allowing them to assemble in a manner similar to natural extra cellular matrix. Example of such extracellular component are; collagens, gelatin, laminin, fibrinogen, and fibronectin; growth factors (insulin like growth factor, platelet derived growth factor, transforming growth factors, fibroblast growth factors, epithelial and endothelial growth factors, angiogenic factors, neuronal growth factor, stem cell growth factor, bone morphogenic proteins) antioxidant enzymes like superoxide dismutase, nutrition transporters like insulin and transferring, small molecules like polyamines (glutathione, spermidine, putresceine, cadaverin). Bone like extracellular mimicry is achieved by adding in the water phase minerals salts like calcium sulfate, tricalcium phosphate, hydroxyapatite, bone putty, and non mineral components of bone like demineralized bone matrix, bone morphogenic proteins.

A total heterogeneous cell population of an organ or a selective population of cells can be incorporated in the emulsion, so as to ensure major ultrastructure of a tissue can be regenerated in the scaffold. Evidence of capability of cells to create tissue like ultrastructures in three dimensional cultures is provided in Seminars in Cell & Developmental Biol. 13, pp. 447-454, 2002.

Tissue like ultrastructure formed by the methods of this invention is supported through formation of vasculature during in vivo incubation period, thereby ensuring oxygen and nutrition delivery and removal of toxic metabolite products (like lactic acid, ammonia, and carbon dioxide) on a long term basis to the cells inside the emulsion scaffold. Similarly tissue like ultrastructure creation during in vitro culture is supported through oxygen and nutrition availability through fluorocarbon and presence of extra cellular matrix components in three dimensions.

This method does not involve any toxic material hence the regulatory issues are expected to be less stringent unlike in case of many other scaffolds.

Collectively these modifications allow direct addition of cells without compromising their functionality by unwanted differentiation and their viability by adequate supply of nutrition and oxygen. Due to the above mentioned innovative advantages it is possible to assemble tissues with realistic dimensions.

### EXAMPLE 1

40 milliliter of perfluorodecalin is mixed gradually with a sterile solution of 10 milliliter of 5% gelatin solution in Earle's balanced salt in the presence of 100 milligram of egg yoke phospholipid and 10 milligram of hyaluronic acid to form a highly viscous emulsion using ultrasound mixing machine. This emulsion is then mixed with 1.0 million CHO-K1 cells suspended in about 0.5 milliliter of Dulbecco's modified Eagle's medium. Cells are gently mixed with emulsion using a sterile flat spatula and swirling motion.

A 0.5 milliliter of 10% solution of microbial transglutaminase in Earle's balanced salt solution is then mixed with 10 mL of emulsion. About 2 mL of emulsion is dropped in 100 milliliter of Dulbecco's modified Eagle's medium containing 5% fetal calf serum in the form of 1-2 millimeter droplets from a 21 gauge hypodermic needle using a 2 mL syringe. These droplets along with the culture media is transferred to a 100 mL spinner bottle and incubated for crosslinking and culture in an incubator at 37° C. and 5% carbon dioxide under intermittent agitation.

After 6 days of culture with half of the medium volume changed every 24 hours, the number of CHO-K1 cells is measured as 30 million cells per milliliter of microcarriers.

All the operations are conducted in a sterile environment using sterile ingredients.

### EXAMPLE 2

A sterile 10 milliliter of 5% gelatin solution in Earle's balanced salt solution is mixed gradually with 40 milliliter of perflurooctane in the presence of 100 milligram of egg yoke phospholipid and 10 milligram of hyaluronic acid to form a thick emulsion. This emulsion is then mixed with 1.0 million liver cells isolate from liver of adult female goat by collagenase treatment suspended in about 0.5 milliliter of Ham's F12 medium with antibiotic. Cells are gently mixed with emulsion using a sterile flat spatula and swirling motion.

A 0.5 milliliter of 10% solution of factor XIII and 10 units of thrombin in Earle's balanced salt solution is then mixed with 10 mL of emulsion. 10 microlitres of this emulsion is quickly coated as a dot of about 2 millimeter diameter and height in each well of 96 well plate using a micropipette. Plates are kept at room temperature in saturated moisture condition and after 30 minutes 100 microlitres of Ham's F12 medium containing 10% fetal calf serum and antibiotics is added on each well for long term culture. After three weeks of culture with 50% change of medium every 24 hrs, cell viability was found to be 95% with MUT assay.

All the operations are conducted in a sterile environment using sterile ingredients.

### EXAMPLE 3

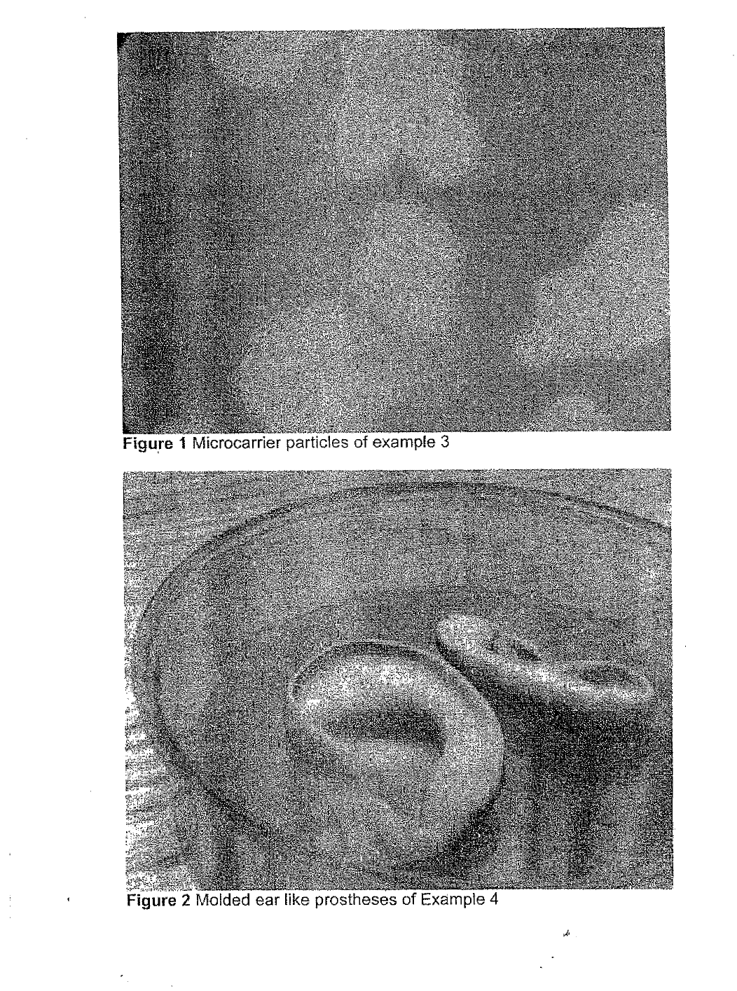

In order to make devices like microcarrier, emulsion of example 1 with transglutaminase but without cells is suspended as small droplets using a 21 gauge needle and syringe in sterile water below 5° C. and left for crosslinking for 10 hours. Afterwards microcarrier is collected on a sieve and dried by washing in ethanol and then under vacuum or by directly by freeze drying. Dried microcarrier particle as observed under optical microscope is shown in FIG. 1.

All the operations are conducted in a sterile environment using sterile ingredients.

### EXAMPLE 4

For making complex shapes using the emulsion of example 2 with transglutaminase and without cells, a mold is made using a block of Teflon. Teflon is preferred for its non adhering properties. Emulsion is filled in the shaped cavity and left for crosslinking overnight. Afterwards shaped object is removed and dried in a similar manner of example 3. An ear like prosthesis is shown in FIG. 2 that is ready for culture with cartilage cells.

All the operations are conducted in a sterile environment using sterile ingredients.

### EXAMPLE 5

Emulsion of example 1 without cells and with transglutaminase is coated on a 96 well plate in the form of dots of about 1-2 millimeter diameter and height. CHO K1 cells are inoculated externally and cultured in vitro for 6 days. Cultured cells are fixed overnight in 0.25% glutaraidehyde for histological examination. In the histological sectioning, using haematoxlin eosin staining, a thick multilayer growth of cells is observed close to the external surfaces of scaffold with fewer cells observed in the interior of the scaffold FIG. 3.

### EXAMPLE 6

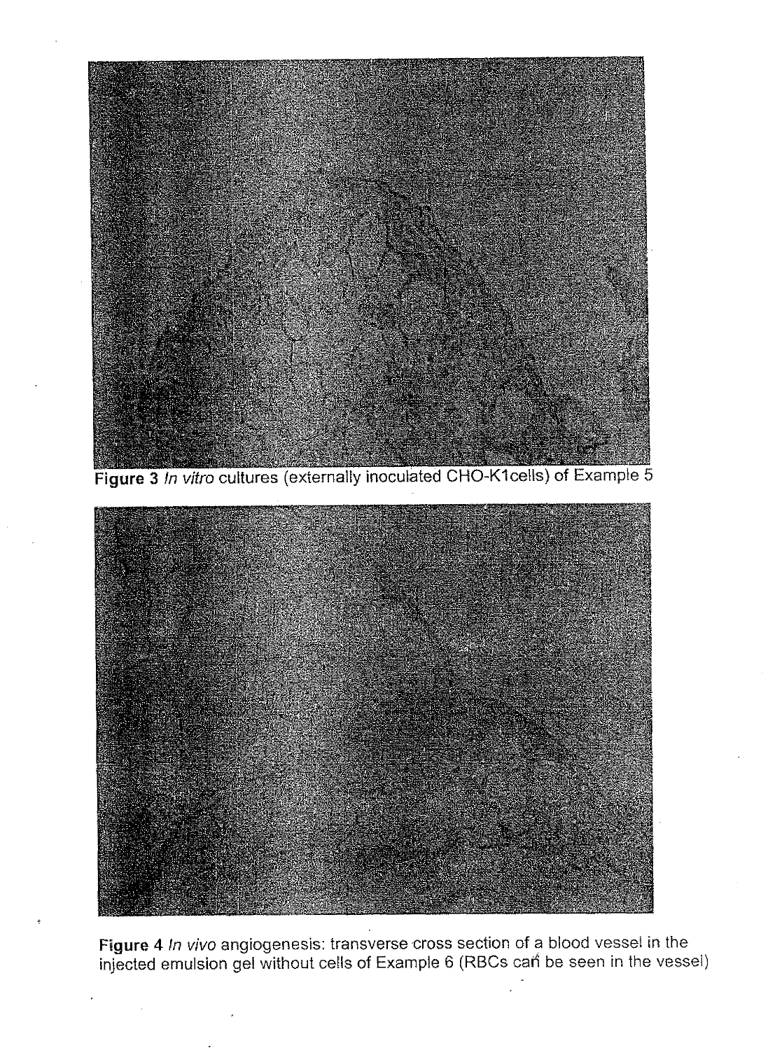

Emulsion of example 1 with transglutaminase and without cells is injected intra peritoneum in three Wistar rats. After two weeks, the injected lump of emulsion is removed surgically and fixed overnight in 0.25% glutaraldehyde for histological examination. In the histological sectioning (FIG. 4) using haematoxlin eosin staining one can observe formation of vasculature containing red blood cells.

### EXAMPLE 7

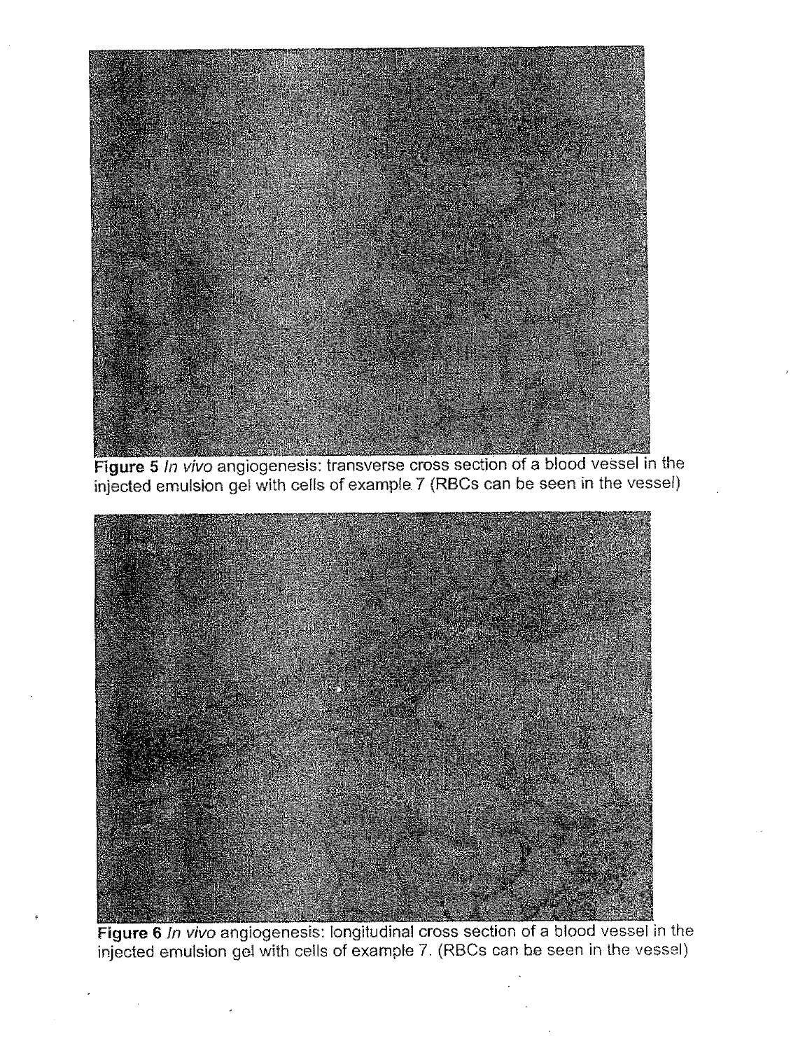

About 3 milliliter of remaining emulsion of example 2 with liver cells is injected intra peritoneum in Wistar rats. After two weeks the injected lump of emulsion is removed surgically and fixed overnight in 0.25% glutaraldehyde for histological examination. In the histological sectioning (FIGS. 5 and 6) several cross-sections of veins and artery like vasculature can be observed apart from other types of cells growing in the pores.

1-11. (canceled)

12. An artificial extra cellular matrix product and artificial human and animal tissue product comprising tissue specific cells.

13. A method for preparing an artificial extra cellular matrix and artificial human and animal tissue product comprising:
making an oil in water emulsion from water solution of extra cellular matrix molecules, other constituents with fluorocarbon and a biocompatible emulsifier;
subjecting the said emulsion to a crosslinking process to form an emulsion gel; and
shaping the emulsion gel to form a device.

14. The method as claimed in claim 13, wherein said extra cellular matrix molecules in pure or crude form are at least one of the following: collagens, gelatin, fibrin, fibrinogen, laminin, glycosaminoglycans their derivative and a part fragment of such molecules.

15. The method as claimed in claim 13, wherein said emulsion comprises water-based physiological buffer solution of extra cellular matrix molecules, the concentration of said molecules being in the range of 1-20% by weight; and an oil phase consisting of one tenth to ten times by volume of the water solution, emulsified within the water solution using a physiological emulsifier and other additives to provide desired physical properties.

16. The method as claimed in claim 13, wherein said oil phase comprises one or more fluorocarbons comprising at least one of the following: perfluarooctane, perfluorohexane, perfluorodecalin, perfluorotripropylamine, perfluoroperhydrophenanthrene, perfluoromethyledecalin, perfluorobutane, octafluoropropane, perfluoropolyethers, hydrofluoropolyethers or silicone oil like polydimethylsiloxane or liquid saturated hydrocarbon like paraffin oil or a mixture of these.

17. The method as claimed in claim 13, wherein said emulsion is prepared in the presence of an emulsifier comprising at least one of the following: bile salts, phospholipids, lecithin, hyaluronic acid, pulmonary surfactant proteins, triglycerides, fatty acids, polyvinyl alcohol, polyvinyl pyrrolidone, pluronic F-68, polypropyleneglycol, carbohydrates or derivatives like alginic acid starch, carboxymethyl cellulose and methyl cellulose, semifluorinated alkanes, fluorinated surfactants, fluorinated amine oxide, polyoxyethylene, polyoxypropylene copolymer (Proxanol 268), pluronic F68, polypropylene oxide and polyethylene oxide surfactants in a concentration of 10 to 100,000 milligram per liter of the emulsion.

18. The method as claimed in claim 13, wherein a crosslinking agent is transglutaminase or lysyl oxidase, and used in a concentration of 0.01% to 1% by weight to the volume of the emulsion for crosslinking the emulsion as permanent gel in physiological conditions and buffer.

19. The method as claimed in claim 13, wherein cells of animal or human tissue/organ are mixed in said emulsion at a concentration of 0.01 million to 100 million per milliliter of emulsion with agitation to disperse cells homogeneously throughout the emulsion.

20. The method as claimed in claim 13, wherein said emulsion gel is shaped into a three dimensional porous scaffold by at least one of the following: casting, molding, extrusion, coating or printing, wherein one or more dimensions of the scaffold is in the range of 0.001 millimeter to 1000 millimeter.

21. The method as claimed in claim 13, wherein said emulsion gel is brought in contact with a sheet of porous polymer film selected from the group consisting of a non-woven fabric, cellulose based papers, screen and meshes made of silicone, biodegradable polymer like poly lactic acid, polyglycolic acid, polyhydroxy butyric acid, poly caprolactone and other natural polymers like alginic acid and chitosan.

22. The method as claimed in claim 13, further comprising injecting or applying the emulsion gel with or without cells in situ in an animal at the site of injury to create artificial tissue with vascularization and sustaining the injected cells alive in artificial extra cellular matrix.

23. The method as claimed in claim 13, further comprising adding desired cells prior to the addition of crosslinking and mixing cells to distribute the cells in the emulsion thoroughly.

24. The method as claimed in claim 15, wherein said oil phase comprises one or more fluorocarbons comprising at least one of the following: perfluorooctane, perfluorohexane, perfluorodecalin, perfluorotripropylamine, perfluoroperhydrophenanthrene, perfluoromethyledecalin, perfluorobutane, octafluoropropane, perfluoropolyethers, hydrofluoropolyethers or silicone oil like polydimethylsiloxane or liquid saturated hydrocarbon like paraffin oil or a mixture of these.

25. A product made in accordance with the method of claim 13.
